(19) United States
(12) Patent Application Publication
(10) Pub. No.: US 2015/0305661 A1
(43) Pub. Date: Oct. 29, 2015

(54) METHOD AND SYSTEM FOR COLLECTING A RETURN SACCADE PARAMETER DURING A VISUAL TEST, AND USE FOR DIAGNOSING AN OCULOMOTOR DISORDER

(71) Applicant: E(YE)BRAIN, Ivry Sur Seine (FR)

(72) Inventor: Magali SEASSAU, PARIS (FR)

(21) Appl. No.: 14/648,589

(22) PCT Filed: Nov. 27, 2013
(86) PCT No.: PCT/FR2013/052879

§ 371 Date: May 29, 2015

(30) Foreign Application Priority Data
Nov. 30, 2012 (FR) 1261521

(51) Int. Cl. A61B 5/16; A61B 3/113

(52) U.S. Cl. CPC A61B 5/16; A61B 3/113

(57) ABSTRACT

Disclosed is a method for collecting data when a patient is subjected to a visual test involving line returns by the patient. The method includes: subjecting the patient to the visual test; recording the eye movements of the patient during the test; determining, in the records obtained, return saccades that make up line returns; calculating return saccade parameters from the records that correspond to the determined return saccades. The parameters can include an average number of saccades over a plurality of line returns, an average of ratios over the plurality of line returns, each ratio corresponding to the ratio between the amplitude of the first time-based return saccade in the line return and the total amplitude of the return saccades forming the same line return. The method is useful for diagnosing an oculomotor disorder that is involved in dyslexia.

### FIELD OF THE INVENTION

The present invention concerns the field of the collection of data when a subject is submitted to a visual test involving at least one line feed return by at least one eye of the subject. More particularly, the invention concerns a method directed to the collection of such data, a corresponding system, as well as the use of such a system to diagnose an oculomotor disorder, preferably an oculomotor disorder involved in dyslexia.

### CONTEXT OF THE INVENTION

The method and the system may in particular assist in the detection of a oculomotor disorder or abnormality in a subject, liable to be at the origin of dyslexia.

An oculomotor disorder, that is to say an abnormality in the movement of the eyes, may appear in certain diseases, for example strabismus or surface dyslexia which is a main type of dyslexia, another main form of dyslexia being phonological dyslexia whereby a subject confuses letters or sounds.

Surface dyslexia may incorporate visual dyspraxia, that is to say poor binocular coordination by a subject. Binocular coordination reflects the right-left coordination between the subject's two eyes. In other words, it is the relative movement of the two eyes during the same task, also called vergence, which, in the case of dyspraxia, introduces a spatial offset between the two eyes which may be measured in degrees.

The publications “Dyslexic children are confronted with unstable binocular fixation while reading” (S. Jainta et al., 2011) (see page 1, right hand column, lines 3 to 15) and “The binocular coordination of eye movements during reading in children and adults”, H. Blythe et al., 2006 (see section 1.1 and p.3899, right hand column, lines 49 to 55) make use of an analysis of this binocular coordination in reading tests.

Numerous tests have been established to attempt to detect surface dyslexia in young children as early as possible.

The works of Rayner (1978) and Pavlidis (1981) have in particular shown that children suffering from such dyslexia presented serious reading problems, in particular characterized by a high number of regressive fixations (that is to say towards the left for a text reading from left to right, as opposed to progressive fixations), by shorter progressive saccades (saccades representing the movement of the eyes from one word to another) and by longer fixations than normal readers of the same age.

Reading tests have thus been developed, as referred to in those two aforementioned publications, and the exploitation of parameters has been refined.

Similarly, visual search tests have been developed, in particular in relation to the occurrences of a letter within a series of letters, as referred to in the publications “The eye movements of dyslexic children during reading and visual search: Impact of the visual attention span” by C. Prado, M. Dubois and S. Valdois (Vision Research 47 pp. 2521-2530, 2007) and “Immaturity of binocular saccade coordination in dyslexic children: evidence from a reading and visual search study” by Maria Pia Bucci, Naziha Nassibi, Christophe-Loic Gerard, Emmanuel Bui-Quoc, Magali Seassau (PlosOne 7(3), e33458), on the basis of which parameters have been exploited.

These tests are visual tests involving at least one line feed return by at least one of the subject's eyes, and generally several line feed returns. The reading test consists of reading a series of symbols, generally typographical, distributed over several horizontal lines whereas the visual search test consists of searching for and finding the occurrences of a defined symbol within the series of symbols.

In these tests, the subject goes through or scans the symbols line by line. A line feed return thus corresponds to the movement of the eye between an end of a line of symbols and the start of the following line of symbols, in the direction of going through or scanning of the lines.

In the publication “The eye movements of dyslexic children during reading and visual search: Impact of the visual attention span” by C. Prado, M. Dubois and S. Valdois (Vision Research 47 pp. 2521-2530, 2007), a reading test of francophone text and a visual search test for a letter within a paragraph lacking semantic content were performed on child subjects to determine the influence of certain parameters in the detection of the dyslexia. The total number of fixations, the average time of fixation, the percentage and the time of the regressive fixations, and the number and the time of the progressive fixations were calculated from recordings of the movements of the right eye of the subjects.

Sometimes the parameters used in these publications do not identify certain dyslexic subjects with certitude.

### SUMMARY OF THE INVENTION

The inventors have found that the use of one or more other well-chosen parameters makes it possible to improve the identification of dyslexic subjects by visual testing, of reading task or visual search task type.

In this context, the invention is directed more particularly to a method, for example for collecting data, comprising the following steps:

submitting the subject to a visual test involving at least one line feed return by at least one of the subject's eyes;

during that task, recording the movements of the subject's eye using an eye movement tracking device;

determining, using a digital processing device, in the recordings obtained, return sweep saccades composing all or part of the at least one line feed return of the subject's eye;

calculating at least one return sweep saccade parameter based on the recordings corresponding to the determined return sweep saccades.

Parameters relative to the return sweep saccades do indeed enable cases of oculomotor disorder to be revealed where the conventional parameters are insufficient. They thus prove to be a precious aid in the diagnosis of dyslexia in particular.

Such a return sweep saccade parameter may be obtained by processing the movement recordings of one or both of the subject's eyes, during the visual test or later in the absence of the subject, based on the measurements made earlier.

One or more new return sweep saccade parameters may be used in isolation or in combination with one or more conventional parameters.

Thus, in an extension to the invention, this data collection method may be used in a method of detecting an oculomotor abnormality in a subject in which, for example, in addition to the preceding steps, the calculated parameter is compared with at least one reference threshold value. In other words, the determined parameter is compared with at least one threshold value to determine an oculomotor abnormality.

This detection method makes it possible to assist in the identification of subjects presenting an oculomotor abnormality, for example, on account of dyslexia or strabismus, by the use of a visual test of reading or visual search type.

In a complementary manner, the invention is directed to a system comprising:

a test module for submitting the subject to a visual test involving at least one line feed return by an eye of the subject;

an eye tracking module configured to record, during said task, the movements of at least one eye of the subject;

a digital processing module configured for determining, in the recordings obtained, return sweep saccades composing all or part of the at least one line feed return of the subject's eye;

a module for calculating at least one return sweep saccade parameter based on the recordings corresponding to the return sweep saccades determined.

By extension, this system, which is dedicated to the collection of data, may be included in a system for detecting an oculomotor abnormality in a subject in which, for example, in addition to these modules, the detecting system also comprises a module for comparing the calculated parameter with at least one reference threshold value.

The system according to the invention has similar advantages to those of the method set forth above, in particular that of providing one or more new parameters improving the detection of an oculomotor disorder, for example linked to dyslexia, during visual tests.

Optional features of the method according to the invention are furthermore defined in the dependent claims. The system according to the invention may also comprise means configured to implement these optional features.

In particular different types of return sweep saccade parameters may be provided.

In an embodiment, the at least one return sweep saccade parameter depends on a number of return sweep saccades constituting the same line feed return.

In particular, the at least one return sweep saccade parameter may comprise an average number of return sweep saccades over a plurality of line feed returns.

As a matter of fact, the inventors have found an average increase in the saccades constituting the line feed returns in certain subjects affected by oculomotor disorders. The return sweep saccade parameters so defined prove particularly effective in the diagnosis of such disorders, of dyslexia type.

In another embodiment, the at least one return sweep saccade parameter depends on a ratio between a horizontal amplitude of a given return sweep saccade in a line feed return and a total horizontal amplitude of return sweep saccades constituting the same line feed return.

The concept of horizontality is to be put into relation with the horizontality of a line. Thus, the horizontal amplitude corresponds to the path of the saccade considered along a line (thus the path of the eye along that line). What is important is to take the amplitude according to the orientation of the line and thus of the line feed return.

The total horizontal amplitude is generally close to or equal to the length of the line since the eyes of the subject return to the start of the following line, in a line feed return. Sometimes however, return sweep saccades that are parasitic, in that they are regressive (that is to say in the opposite direction to the line feed return), may occur increasing the total horizontal amplitude, here represented by the sum of the amplitudes (absolute) of the return sweep saccades constituting the line feed return.

The ratio defined above thus represents the portion of the line passed along by the return sweep saccade considered and thus the accuracy of that return sweep saccade in the line feed return.

In particular, the given return sweep saccade is preferably the first temporal saccade within the line feed return. In other words, it is the return sweep saccade which commences at the end of the line which has just been passed along for the test.

The inventors have found that this ratio, as return sweep saccade parameter, also proves to be a good indicator of oculomotor disorders in certain subjects. As a matter of fact, the inventors have found that the first temporal saccade is generally less effective in a subject affected by a oculomotor disorder than in a healthy subject.

According to another particular feature, the at least one return sweep saccade parameter comprises an average of ratios over a plurality of line feed returns, each ratio for a line feed return corresponding to the ratio between a horizontal amplitude of the first temporal return sweep saccade in said line feed return and a total horizontal amplitude of return sweep saccades constituting the same line feed return.

This configuration makes it possible to obtain a return sweep saccade parameter which is consolidated, since established over several lines for the same subject. The assistance in detecting an oculomotor disorder is thus increased.

In a particular embodiment directed to the combination of the aforementioned parameters, the at least one return sweep saccade parameter combines an average number of return sweep saccades over a plurality of line feed returns and an average of ratios over the plurality of line feed returns, each ratio for a line feed return corresponding to the ratio between a horizontal amplitude of the first temporal return sweep saccade in said line feed return and a total horizontal amplitude of return sweep saccades constituting the same line feed return.

As shown below, the combined use of these return sweep saccade parameters averaged over several lines is particularly effective for the detection of an oculomotor disorder in a subject.

In particular, the method may comprise the identification of an oculomotor abnormality when the average number of return sweep saccades is greater than a first threshold value or when the average of ratios is less than a second threshold value.

In an embodiment which may be combined with the preceding embodiments, the movements of both the subject's eyes in which return sweep saccades are determined are recorded, and the return sweep saccade parameter comprises at least one parameter of binocular disconjugacy in amplitude representing a difference in horizontal movement between the subject's two eyes in at least one determined return sweep saccade.

This additional saccadic disconjugacy parameter further improves the detection an oculomotor disorder in a subject.

The inventors have indeed found that binocular coordination in dyslexic subjects is altered in saccade phases, in particular during a line feed return.

All the return sweep saccade parameters referred to previously are determined from the recordings obtained.

As briefly introduced already, the visual test may comprise a task of reading a series of symbols distributed over several horizontal lines or a visual search task for occurrences of a symbol within a series of symbols. Advantageously they are typographical symbols enabling lines of words to be constituted.

An embodiment combining these two tasks may thus provide that the subject is submitted to a task of reading a series of symbols distributed over several horizontal lines and to a task of visual search for occurrences of a symbol within a series of symbols distributed over several horizontal lines; and

the method comprises the identification of an oculomotor abnormality when at least one parameter among

an average number of return sweep saccades over a plurality of line feed returns in the reading task,

an average of ratios over the plurality of line feed returns in the reading task, each ratio for a line feed return corresponding to the ratio between a horizontal amplitude of the first temporal return sweep saccade in said line feed return and a total horizontal amplitude of the return sweep saccades constituting the same line feed return,

an average number of return sweep saccades over a plurality of line feed returns in the visual search task,

an average of ratios over the plurality of line feed returns in the visual search task, each ratio for a line feed return corresponding to the ratio between a horizontal amplitude of the first temporal return sweep saccade in said line feed return and a total horizontal amplitude of the return sweep saccades constituting the same line feed return,

exceeds a corresponding threshold value, that is to say a threshold value for each type of parameter used above.

These four parameters, which may be complemented by the parameter of binocular disconjugacy in amplitude referred to above, may be associated within the same recording as a result of the collection method defined above.

A main application of the invention is the detection of dyslexia, in particular surface dyslexia, in subjects, in particular children.

The collection of return sweep saccade parameters according to the invention may also be implemented in a context of evaluation of curative methods or systems directed to treating and reducing oculomotor disorders in subjects already diagnosed as dyslexic.

### BRIEF PRESENTATION OF THE DRAWINGS

Still other particularities and advantages of the invention will appear in the following description, illustrated by the accompanying drawings, in which:

FIG. 1 diagrammatically illustrates a system for assisting in the detection of an oculomotor abnormality by visual search test, according to the invention;

FIG. 2 represents an example paragraph for a visual test of reading type (on the left) and of visual search type (on the right) adapted for children aged from 8 to 9;

FIG. 3 represents an example paragraph for a visual test of reading type (on the left) and of visual search type (on the right) adapted for children aged from 10 to 11;

FIG. 4 represents another example paragraph for a visual test of reading type (on the left) and of visual search type (on the right) adapted for children aged from 12 to 13;

FIG. 5 illustrates a recording of the movements of the eyes of a healthy subject during a visual test;

FIG. 6 illustrates a recording of the movements of the eyes of a dyslexic subject during the same visual test;

FIG. 7 shows a summary table of the results of visual tests on a group of child subjects aged between 8 and 9;

FIG. 8 shows a summary table of the results of visual tests on a group of child subjects aged between 10 and 11;

FIG. 9 shows a summary table of the results of visual tests on a group of child subjects aged between 12 and 13;

FIGS. 10a and 10b respectively illustrate the behavior of the average number of return sweep saccades and the behavior of the average gain of the first temporal return sweep saccade in tests of reading and visual search, between the dyslexic subjects of all groups and the healthy subjects; and

FIGS. 11a and 11b respectively illustrate the behavior of the average number of return sweep saccades and the behavior of the average gain of the first temporal return sweep saccade in tests of reading and visual search, between the dyslexic subjects and the healthy subjects taken by age group.

### DETAILED DESCRIPTION OF EMBODIMENTS OF THE INVENTION

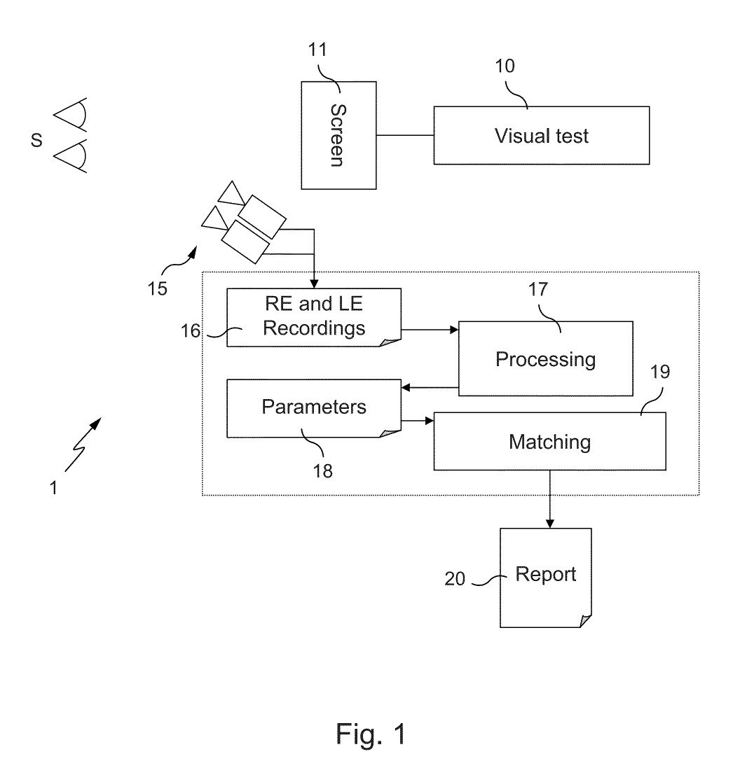

In the embodiment of FIG. 1, a system 1 for assisting in the detection of an oculomotor abnormality comprises a test part and an acquisition and processing part.

The test part comprises in particular a test module 10 for subjecting the subject S to a visual test involving at least one line feed return by at least one eye of the subject, including a task of reading a series of symbols distributed over several horizontal lines and/or a task of visual search for occurrences of a symbol within the series of symbols, as well as a screen 11 for displaying that series of symbols to the subject at a distance comprised between approximately 45 and 65 cm preferably approximately 60 cm.

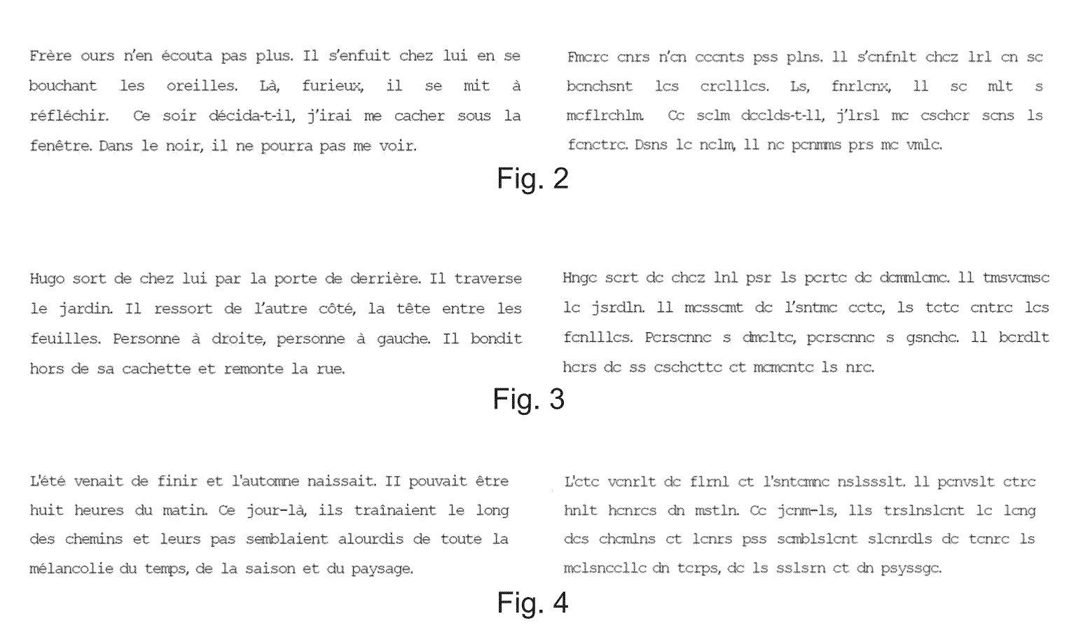

FIGS. 2 to 4 illustrate examples of paragraphs presented to the subject for the visual test.

The paragraphs that are given a semantic content on the left of those Figures are dedicated to the reading task, while the paragraphs situated on the right of those Figures are dedicated to the visual search task incorporating only consonants without semantic content.

In the experiments conducted and presented subsequently, a paragraph selected according to the test to perform and the age of the subject S is displayed in a field of vision of the subject S with a width comprised between 20 and 40 degrees, preferably between 25 and 30 degrees, advantageously of 29 degrees and a height comprised between 5 and 10 degrees, advantageously of 6.4 degrees.

The texts presented to the subject advantageously comprise several lines, for example between 2 and 10 lines, in particular between 3 and 5 lines.

In the example of the Figures, these texts comprise 4 lines, together formed by approximately 40 words and 180 characters. Thus the eyes of the subject are caused to perform three line feed returns. In practice, tests are provided involving between 1 and 10 line feed returns, preferably between 2 and 6 line feed returns, for example 3 or more, in particular 4 or 5.

Each line may comprise between 30 and 100 symbols (here typographical characters) so as to form between 7 and 20 words. Thus the subject is caused to perform several horizontal progressive saccades to go through or scan the line and return sweep saccades to pass from one line to another.

The test module 10 instructs the subject S either to read the text for the reading task, or to search for a letter, for example the letter “r”, in the paragraph displayed for the visual search task.

The subject may be caused to successively perform both tasks.

Returning to FIG. 1, the acquisition and processing part comprises a video acquisition module 15 placed facing the subject's eyes to acquire, with a high cadence (for example every 4 ms), the movement of each of the eyes during the visual test.

The video acquisition module 15 performs pre-processing of the acquired images, according to conventional image processing techniques, in order to obtain a recording, over time, of the movement of each eye in amplitude along the horizontal axis (degrees relative to the center of vision).

The video acquisition module 15 performs the eye movement tracking for both eyes simultaneously. To that end, the Mobile Eyebrain Tracker (Mobile EBT®) system may be used.

The two curves 16 obtained are then recorded in the system to be processed immediately or later upon collection of the data.

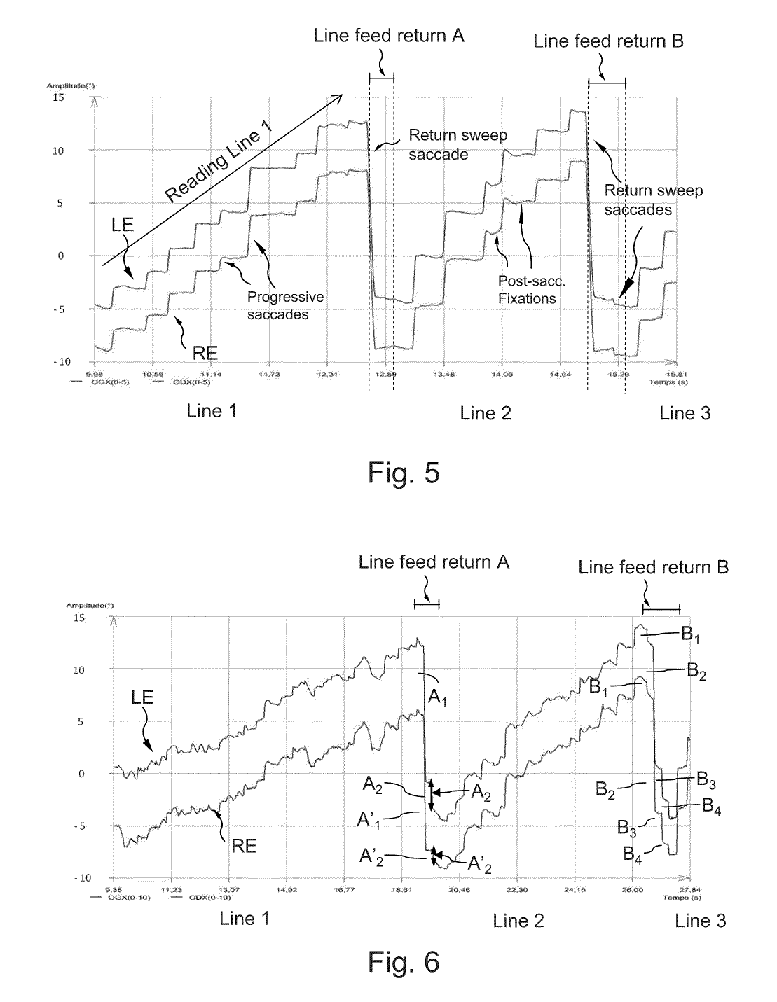

FIG. 5 represents a portion of the recordings for the right eye RE (lower curve) and for the left eye LE (top curve), for a subject not affected by dyslexia, in a reading test (FIG. 2, 3 or 4). The recordings on reading two lines and the start of a third line are represented.

The acquisition and processing part of the system 1 also comprises a digital processing module 17 configured to determine, in the recordings 16, return sweep saccades composing all or part of at least one line feed return of the subject's eye and to calculate at least one return sweep saccade parameter 18 from the recordings corresponding to the return sweep saccades determined.

This module 17 in particular performs the identification of saccades and post-saccadic fixations in the recordings 16, and in particular the identification of saccades composing line feed returns.

As described in the C. Prado et al. publication, a saccade may be identified at the locations where the movements of the eye present a velocity peak greater than 30°/s and less than 800°/s and an amplitude of movement greater than 0.5 or 1 degree relative to the position of fixation prior to the triggering of the saccade. The start and the end of the saccade may be delimited by the change in sign of the velocity at two rearward points. Of course other criteria may be employed, in particular, for example, an algorithm wherein the detection threshold of the velocity is dynamic, that is to say averaged per subject in general for a signal duration less than 20 seconds. The velocity is then calculated, preferably, at 3 centered points.

In the example of FIG. 5, saccades are thus identified between the times 9.98 s and 12.31 s corresponding to progressive saccades made during the reading of a first line. Similarly. the saccades between the times 12.98 s and 14.80 s correspond to progressive saccades made during the reading of a second line. It is to be observed that each saccade represents a significant movement of the eyes of approximately +1 to +5 degrees (transition zone between two horizontal plateaus). The detection of the progressive saccades and post-saccadic fixations (periods between two saccades, that is to say the horizontal plateaus in the Figure) is not necessary for the purposes of the present invention.

Return sweep saccades are also identified in the recording. The identification of these saccades relative to the progressive saccades may rely on the fact that they have an inverse amplitude (negative here). Furthermore to identify them relative to regressive saccades (not shown) made on reading the line, a line feed return is characterized by a first regressive saccade not followed by a relatively long period of fixation (a fixation is defined by a plateau on the curve. They are, in the reading, of a duration varying from 100 to 600 ms), this first saccade being situated at a line end.

The first saccade may be of high amplitude in absolute value (here −17 degrees compared with the +1 to +5 of the saccades in line reading). Thus a threshold for example set at 10 degrees for a test presented over 29 degrees (i.e. a ratio of ⅓) could enable a first return sweep saccade to be detected, in an embodiment.

The saccades of negative amplitude following that first saccade are also return sweep saccades constituting the same line feed return, until the subject's eyes attain the angular position corresponding to a line beginning (−9 degrees approximately in the Figure).

All the successive saccades so measured, not followed by a relatively long period of fixation (greater than 100 ms for example) and/or preceeding a reading progressive saccade, are considered as forming part of the same line feed return.

In the example represented in the Figure, the first line feed return is constituted by a single return sweep saccade, and the second line feed return is constituted by two return sweep saccades.

Once the line feed returns and their return sweep saccades have been identified, the processing module 17 determines and calculates in particular one or more return sweep saccade parameters as described below. Possibly other conventional parameters may also be calculated to be used in combination with that or those return sweep saccade parameters in the context of detecting oculomotor disorders.

Lastly, the acquisition and processing part of the system 1 comprises a module 19 for comparing the value or values determined for the return sweep saccade parameters 18 with at least one of the corresponding threshold values TV so as to determine an oculomotor abnormality in the subject tested.

This module may consist for example in issuing an oculomotor report 20 on which appear the various parameter or parameters determined by the module 17, as well as the corresponding threshold value or values.

As a variant, this module may make a digital comparison between the value or values of the parameters considered and their corresponding threshold value to issue decision information of “dyslexic subject” or “non-dyslexic subject” type.

FIG. 6 represents a portion of the recordings for the right eye RE (lower curve) and for the left eye LE (top curve), for a subject affected by dyslexia, in the same reading test (FIG. 2, 3 or 4). The recordings on reading the three lines are represented in the Figure. Two line feed returns, denoted A and B, are thus identified by the module 17 within that recording.

The module 17 determines in particular all or some of the parameters described below for a plurality of line feed returns. These same parameters may be determined when a single line feed return is available in the recordings 16.

Some parameters are calculated for a given eye (monocular parameters). A similar calculation may thus be carried out for the other eye in order to have a larger amount of information available. For the following part of the description, reference is made to a single eye, for example LE. Of course, the teachings which follow may thus be applied for the other eye as a variant or in combination.

These monocular parameters are based on the recording 16 of a single eye comprising:
an average number of return sweep saccades over a plurality of line feed returns in the reading task,
an average of ratios over the plurality of line feed returns in the reading task, each ratio for a line feed return corresponding to the ratio between the horizontal amplitude of the first temporal return sweep saccade in said line feed return and the total horizontal amplitude of the return sweep saccades constituting the same line feed return,

The same parameters may be obtained when the subject is submitted to the visual search test (texts on the right in FIGS. 2, 3 and 4):
an average number of return sweep saccades over a plurality of line feed returns in the visual search task,
an average of ratios over the plurality of line feed returns in the visual search task, each ratio for a line feed return corresponding to the ratio between the horizontal amplitude of the first temporal return sweep saccade in said line feed return and the total horizontal amplitude of the return sweep saccades constituting the same line feed return,

In the example of FIG. 6, the module 17 has identified two return sweep saccades, denoted A1 and A2, constituting the first line feed return A, and identifies four return sweep saccades, denoted B1, B2, B3 and B4, constituting the second line feed return B.

The number N_{A} of return sweep saccades for the first line feed return A is thus 2, and the number N_{B} of return sweep saccades for the second line feed return B is 4. Other line feed returns L (not shown) may also have their own number N_{L} of return sweep saccades.

The average number Nₐᵥ of return sweep saccades over a plurality of line feed returns in the visual test (reading or visual search) is thus the average of these numbers N_{L} of return sweep saccades, i.e. Nₐᵥ=(ΣN_{L})/(number of line feed returns).

In the example of the Figure, Nₐᵥ=(N_{A}+N_{B})/2=3.

The module 17 also determines the horizontal amplitude of each return sweep saccade Aᵢ, Bⱼ, etc. To simplify the explanations, A₁ is used to denote the horizontal amplitude of the return sweep saccade A₁, A₂ that of the return sweep saccade A₂, etc. These horizontal amplitudes are expressed in degrees and correspond to the movement of the eye in the return sweep saccade (vertical movement on the recordings of the Figure).

Next the module 17 calculates the gain G_{L} of each line feed return L. The gain G_{L} is the ratio between the horizontal amplitude of the first temporal return sweep saccade L₁ and the total horizontal amplitude of the line feed return, for example the sum of the horizontal amplitudes of return sweep saccades constituting the line feed return.

In a variant, the total horizontal amplitude may be calculated as the amplitude between the two extreme positions of the same eye (that is to say the two points that are the highest and the lowest on the LE curve of the Figure).

In the example of the Figure, G_{A}=A₁/(A₁+A₂) and G_{B}=B₁/(B₁+B₂+B₃+B₄)

The average Gₐᵥ of the ratios (or gains) over the plurality of line feed returns in the visual test is thus the average of the gains G_{L} calculated for all the line feed returns considered: Gₐᵥ=(ΣG_{L})/(number of line feed returns). In the example of the Figure Gₐᵥ=(G_{A}+G_{B})/2.

In addition to these monocular return sweep saccade parameters, a binocular parameter is also calculated by module 17. This binocular parameter is a parameter of binocular disconjugacy in amplitude representing a difference in horizontal movement between the two eyes of the subject in at least one determined return sweep saccade.

Let A′₁ denote the horizontal amplitude of the first temporal return sweep saccade for the right eye RE in the line feed return A (A₁ is the amplitude for the LE for the same first temporal return sweep saccade), A′₂ that of the second return sweep saccade for RE in the same line feed return A, etc.

It should be noted that it is possible for one eye to make more return sweep saccades than the second. It has however been observed that these additional return sweep saccades are generally of very small amplitudes compared with the main return sweep saccades of the line feed return. Thus, in an embodiment, it is provided to detect/identify solely the return sweep saccades which have an amplitude greater than 1°. By application of this threshold amplitude, both eyes present the same number of return sweep saccades in most situations.

However a second threshold value is applied in the other situations to measure the parameter of binocular disconjugacy. This threshold value corresponds to a temporal threshold of 80 ms, beyond which if a new return sweep saccade has been detected on one eye but not on the other, the return sweep saccade is accounted for as being a return sweep saccade but no binocular disconjugacy parameter is calculated over that particular portion of recording of the movements of the eyes. Once this new return sweep saccade has been processed uniquely as monocular, the detection of another return sweep saccade is resynchronized on both eyes.

The parameter δ of binocular disconjugacy for a return sweep saccade may be calculated by the module 17 as a ratio between the difference in horizontal amplitude between LE and RE in the same return sweep saccade and the horizontal amplitude of that saccade, i.e. δ_{A2}=(A₂−A′₂)/A₂*100.

In this example, the amplitude A₂ is used as the value of the horizontal amplitude of the saccade. As a variant, A′₂ may be taken or an average may be made of the two amplitudes.

Each parameter δ_{Li} of binocular disconjugacy for a return sweep saccade Lᵢ may be used independently of the others in the process of detecting an oculomotor disorder. As a variant, an average parameter δ_{L} of binocular disconjugacy for the whole of a line feed return L may be calculated: δ_{L}=(Σδ_{Li})/N_{L}, where N_{L} is the number of return sweep saccades constituting the line feed return L. In another variant, a global parameter Δ of binocular disconjugacy for all the line feed returns may be calculated: Δ=(ΣΔ_{L})/(number of line feed returns).

A description will now be given of visual search tests carried out on 30 dyslexic children and 42 normal reading children aged 8 to 13, tested according to their age (FIGS. 5 to 11b).

The normal reading children in particular have made it possible to establish normals and corresponding threshold values at p<0.05 (that is to say 5% equivalent to twice the standard deviation at the mean) for all of or some of the parameters mentioned earlier. The invention is not however limited to this type of thresholding, it also being possible to implement other techniques to improve or slacken constraints on the presence of false positives.

The children are distributed into three age groups: 8-9 year olds; 10-11 year olds and 12-13 year olds.

For the children of the 8-9 age group (15 normal reading children and 14 dyslexic children), the visual test is carried out on one of the paragraphs of FIG. 2. For those aged from 10-11 (16 normal reading children and 11 dyslexic children), the visual test is carried out on one of the paragraphs of FIG. 3. Lastly, for those aged from 12-13 (11 normal reading children and 5 dyslexic children), the visual test is carried out on one of the paragraphs of FIG. 4.

The eye movements were recorded by the Mobile EBT® video-oculography system.

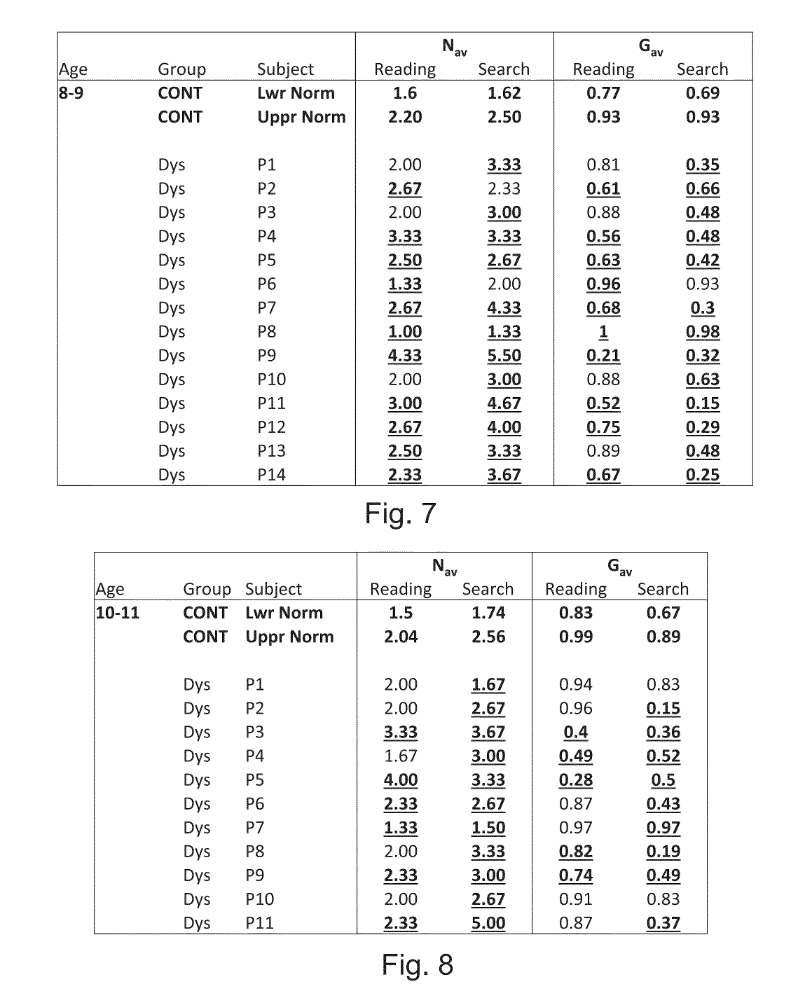

The tables in FIGS. 7, 8 and 9 illustrate the results obtained for the three age groups based on the recordings for LE and RE. The values entered in these tables are in fact averages for LE and RE. Of course, similar tables may be established for RE only and/or for LE only.

Normal reader children (healthy non-dyslexic subjects of each age group) serve as controls for establishing the normals for the aforementioned parameters, as well as the corresponding threshold values (p<0.05), in particular for the lower and upper bounds as adopted in the tables.

These bounds are compared with the return sweep saccade parameters respectively calculated in order to identify a possible oculomotor anomaly.

These results show:
that the dyslexic children make more line feed return sweep saccades (Nₐᵥ) than the healthy children (F(1,66)=25.75, p<0.001), as diagrammatically shown in FIG. 10a. This is true in a reading test (p<0.008) and in a visual search test (p<0.001). It is thus provided that the comparison made by module 19 preferentially determines whether a parameter on the average number of return sweep saccades exceeds the upper bound obtained for the healthy subjects, or possibly the lower bound;
that the dyslexic children have a lower gain Gₐᵥ than the healthy children (F(1.66)=31.39, p<0.0001), as diagrammatically shown in FIG. 10b. This means that the dyslexic children have a first temporal return sweep saccade which is less precise than the healthy subjects. This is true in a reading test (p<0.007) and in a search test (p<0.001). It is thus provided that the comparison made by module 19 preferentially determines whether a parameter on the average gain of the return sweep saccades exceeds the lower bound obtained for the healthy subjects, or possibly the upper bound;

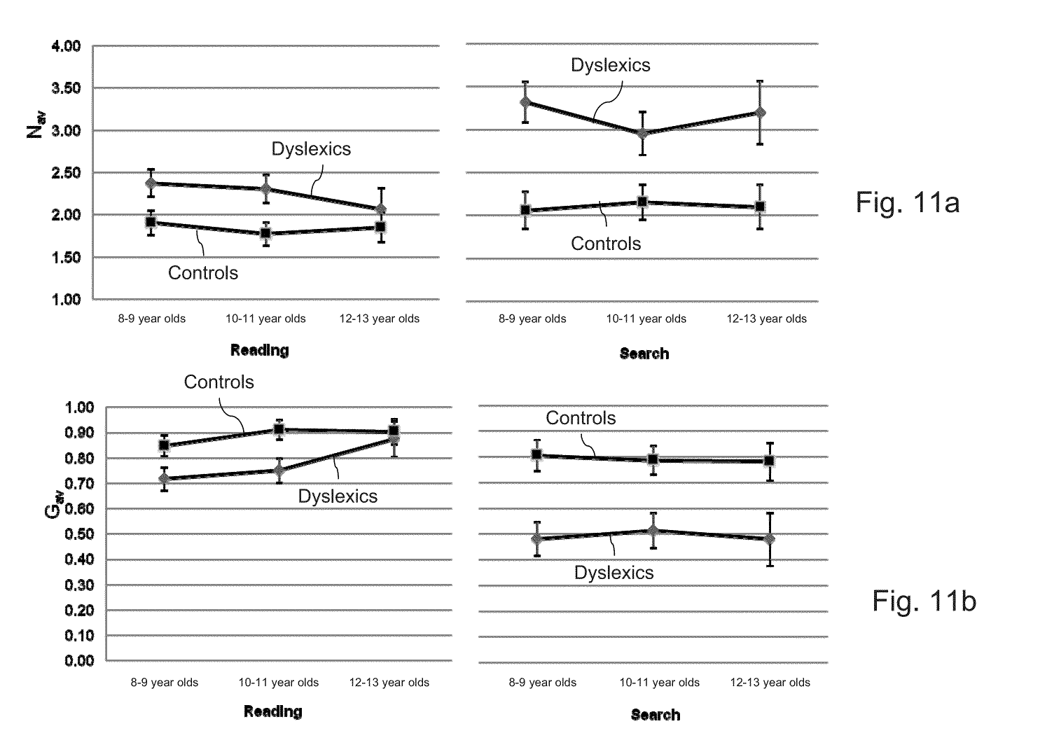

These results are not modulated by age, or on the number of saccades (FIG. 11a, F(2.62)=1.37, p=0.27), or on the gain (FIG. 11b, F<1).

The table of FIG. 7 shows in particular that the entirety of the dyslexic children of the age group 8-9 have at least one monocular line feed return parameter averaged for LE and RE (out of the four defined above) which is deficient (the values in underlined bold are outside the bounds). It should be noted that the majority of the dyslexic children has at least either an Nₐᵥ parameter higher than the upper reference bound, or a Gₐᵥ parameter less than the lower reference bound. However, two dyslexic exceptions exceed the other reference bounds (P6 and P8).

The table of FIG. 8 shows in particular that the entirety of the dyslexic children of the age group 10-11 have at least one averaged monocular line feed return parameter (out of the four defined above) which is deficient (values in underlined bold).

It should be noted that the majority of the dyslexic children has at least either an Nₐᵥ parameter higher than the upper reference bound, or a Gₐᵥ parameter less than the lower reference bound. Two dyslexic exceptions have at least one of their parameters that exceeds the other reference bounds (P1 and P7).

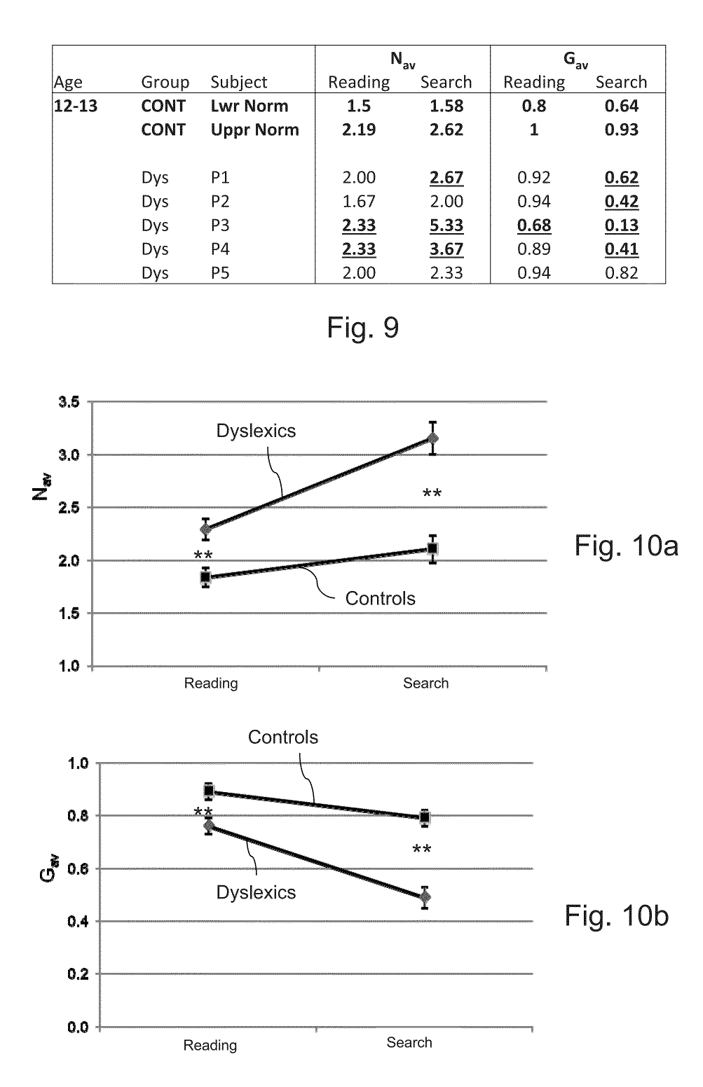

The table of FIG. 9 shows in particular that 80% of the dyslexic children tested in the age group 12-13 have at least one averaged monocular line feed return parameter (out of the four defined above) which is deficient (values in underlined bold).

The combination of the averaged four monocular parameters (Nₐᵥ and Gₐᵥ for each of the two tests) thus makes it possible to detect the dyslexic subjects fairly accurately.

The return sweep saccade binocular parameter (the parameter of binocular disconjugacy in amplitude defined above) may also be used in the detection of an oculomotor disorder by module 19.

The results obtained for the same groups of children show that this parameter of binocular disconjugacy in amplitude is substantially greater in dyslexic subjects than in healthy subjects.

In particular the results obtained show that an average parameter of disconjugacy calculated over all the return sweep saccades of the same line feed return, which average parameter of disconjugacy is equal to 7.4% in healthy subjects in a reading task and equal to 8.9% in the same healthy subjects in a visual search task.

By comparison, that average parameter of disconjugacy is 13.8% in dyslexic subjects in a reading task and 13.3% in the same dyslexic subjects in a visual search task.

Therefore a threshold value for the comparison by the module 19 in order to identify an oculomotor disorder using that parameter of binocular disconjugacy in amplitude may be set at a value comprised between 6% and 11%, for example 8%. Thus, an oculomotor abnormality is detected when that parameter of binocular disconjugacy in amplitude is greater than that threshold value.

The present invention makes it possible to collect and obtain one or more particular parameters relative to return sweep saccades forming line feed returns, which prove effective in the detection and the identification of subjects suffering from an oculomotor abnormality by a simple visual test. In particular, the results obtained based on these parameters show that the dyslexic subjects have a new type of oculomotor disorder characterized by a substantial modification of the number of return sweep saccades or of the average gain in line feed returns, as well as by the presence of a binocular disconjugacy in those saccades.

The preceding examples are only embodiments of the invention which is not limited thereto.

1. A method comprising the following steps:
submitting the subject (S) to a visual test involving at least one line feed return (A, B, L) by at least one of the subject's eyes;
during that task, recording the movements of the subject's eye using an eye movement tracking device (15);
determining, using a digital processing device (17), in the recordings obtained (16), return sweep saccades (A₁, A₂, B₁-B₄, Lᵢ) composing all or part of the at least one line feed return of the subject's eye;
calculating at least one return sweep saccade parameter (18, Nₐᵥ, Gₐᵥ, Δ) based on the recordings corresponding to the determined return sweep saccades.

2. A method according to claim 1, wherein the at least one return sweep saccade parameter depends on a number of return sweep saccades (N_{A}, N_{B}, N_{L}) constituting the same line feed return (A, B, L).

3. A method according to claim 2, wherein the at least one return sweep saccade parameter comprises an average number of return sweep saccades (Nₐᵥ) over a plurality of line feed returns.

4. A method according to claim 1, wherein the at least one return sweep saccade parameter depends on a ratio (G_{A}, G_{B}, G_{L}) between a horizontal amplitude of a given return sweep saccade in a line feed return and a total horizontal amplitude of return sweep saccades constituting the same line feed return.

5. A method according to claim 4, wherein the given return sweep saccade is the first temporal saccade (A₁, B₁, L₁) within the line feed return.

6. A method according to claim 4, wherein the at least one return sweep saccade parameter comprises an average (Gₐᵥ) of ratios over a plurality of line feed returns, each ratio (G_{A}, G_{B}, G_{L}) for a line feed return corresponding to the ratio between a horizontal amplitude of the first temporal return sweep saccade in said line feed return and a total horizontal amplitude of return sweep saccades constituting the same line feed return.

7. A method according to claim 1, wherein the at least one return sweep saccade parameter combines an average number of return sweep saccades (Nₐᵥ) over a plurality of line feed returns and an average (Gₐᵥ) of ratios over the plurality of line feed returns, each ratio for a line feed return corresponding to the ratio between a horizontal amplitude of the first temporal return sweep saccade in said line feed return and a total horizontal amplitude of return sweep saccades constituting the same line feed return.

8. A method according to claim 7, comprising the identification of an oculomotor abnormality when the average number of return sweep saccades is greater than a first threshold value (TV) or when the average of ratios is less than a second threshold value (TV).

9. A method according to claim 1, wherein the movements of both the subject's eyes in which return sweep saccades are determined are recorded, and the return sweep saccade parameter comprises at least one parameter of binocular disconjugacy in amplitude (δ_{A2}, δ_{L}, Δ) representing a difference in horizontal movement between the subject's two eyes in at least one determined return sweep saccade.

10. A method according to claim 1, wherein the visual test comprises a task of reading a series of symbols distributed over several horizontal lines or a visual search task for occurrences of a symbol within a series of symbols.

11. A method according to claim 1, comprising comparing the calculated parameter with at least one reference threshold value.

12. A method according to claim 1, wherein the subject (S) is submitted to a task of reading a series of symbols distributed over several horizontal lines and to a task of visual search for occurrences of a symbol within a series of symbols distributed over several horizontal lines; and
the method comprising the identification of an oculomotor abnormality when at least one parameter (18) among
an average number of return sweep saccades (Nₐᵥ) over a plurality of line feed returns in the reading task,
an average (Gₐᵥ) of ratios over the plurality of line feed returns in the reading task, each ratio for a line feed return corresponding to the ratio between a horizontal amplitude of the first temporal return sweep saccade in said line feed return and a total horizontal amplitude of the return sweep saccades constituting the same line feed return,
an average number of return sweep saccades (Nₐᵥ) over a plurality of line feed returns in the visual search task,
an average (Gₐᵥ) of ratios over the plurality of line feed returns in the visual search task, each ratio for a line feed return corresponding to the ratio between a horizontal amplitude of the first temporal return sweep saccade in said line feed return and a total horizontal amplitude of the return sweep saccades constituting the same line feed return,
exceeds a corresponding threshold value (TV).

13. A system (1) comprising:
a test module (10) for submitting the subject (S) to a visual test involving at least one line feed return (A, B, L) by at least one eye of the subject;
an eye tracking module (15) configured to record, during said task, the movements of at least one eye of the subject;
a digital processing module (17) configured for determining, in the recordings obtained (16), return sweep saccades (A₁, A₂, B₁-B₄, Lᵢ) composing all or part of the at least one line feed return of the subject's eye;
a module for calculating at least one return sweep saccade parameter (18, Nₐᵥ, Gₐᵥ, Δ) based on the recordings corresponding to the return sweep saccades determined.

14. A method for diagnosing an oculomotor disorder, comprising:
providing the system of claim 13; and
testing at least one eye of the subject using said system.

15. A method according to claim 5, wherein the at least one return sweep saccade parameter comprises an average (Gₐᵥ) of ratios over a plurality of line feed returns, each ratio (G_{A}, G_{B}, G_{L}) for a line feed return corresponding to the ratio between a horizontal amplitude of the first temporal return sweep saccade in said line feed return and a total horizontal amplitude of return sweep saccades constituting the same line feed return.
